(19) United States
(12) Patent Application Publication
(10) Pub. No.: US 2004/0116798 A1
(43) Pub. Date: Jun. 17, 2004

(54) Method and system for investigation of central nervous system drugs using 3-D brain source localization

(76) Inventors: Robert Cancro, MT. Kisco, NY (US); Jorge Costa e Silva, New York, NY (US)

Correspondence Address: Oleg F. Kaplun, Esq., Fay Kaplun & Marcin, LLP, Suite 702, 150 Broadway, New York, NY, 10038, US

(21) Appl. No.: 10/317,238

(22) Filed: Dec. 11, 2002

(51) Int. Cl. A61B 5/05

(52) U.S. Cl. 600/409

(57) ABSTRACT

A method for determining effects of a drug on a central nervous system (CNS) of a subject, comprises the steps of: detecting and locating three-dimensional (3-D) brain source regions of first brain waves of the subject using a functional neuroimaging system, the first brain waves being indicative of a CNS disorder and comparing the first brain waves at each of the regions to control data corresponding to brain wave activity of corresponding brain regions in the absence of the CNS disorder. After these steps the drug is administered to the subject and second brain waves of the subject are measured from the brain source regions and compared to one of the first brain waves and the control data. The drug's effectiveness in regard to the CNS disorder is then determined based on the comparison.

### FIELD OF THE INVENTION

The present invention relates to medical and pharmaceutical investigations and more specifically to investigations of the effectiveness of drugs targeted toward disorders of the central nervous system (CNS), primarily psychiatric disorders.

### BACKGROUND INFORMATION

At the present time, the investigation of new pharmaceutical compounds involves testing the compounds first for safety and then for effectiveness. The accepted testing procedure for effectiveness of a drug is the “double blind” procedure. In that procedure, a statistically random sample of subjects is divided into two groups with the subjects of each group being matched by gender, age, etc. to each other. A first one of the groups receives the drug while second group receives a placebo (i.e., sugar pill), without the subject or the physician knowing which pill (or injection etc.) the subjects are receiving. The subjects are then examined for the effects, if any, of the symptoms of the medical problem toward which the drug is targeted. In the case of drugs directed toward the central nervous system (CNS), the symptoms may often not be objectively measured. For example, the effectiveness of drugs intended to relieve depression may me judged based on how the patient feels, but the expressed change in the feelings of patients may be inaccurate.

The effectiveness of drugs often varies considerably from patient to patient. Some subjects respond differently to a drug because of age, sex, gender, metabolism, genetic make-up and other factors, some of which are not completely understood. This is particularly true for drugs directed to the CNS, and especially for drugs directed toward helping cure and/or ameliorate psychiatric disorders. It has been reported, for example, that as many as 30% of patients diagnosed with depression do not respond favorably to their anti-depressive drug regimen.

This field suffers from diagnostic heterogeneity; as the diagnosis of patients is often based on their phenomenology (i.e., a description of specific symptom). This problem of non-effectiveness of CNS disorder drugs is entirely separate from the problem of adverse side effects of many of those drugs.

The effectiveness of CNS drugs has traditionally been measured by their ability to suppress specific symptoms. In general, there has been little attempt, in CNS drug development, to target the reversal of the pathophysiology (i.e. to cure the underlying brain disorder giving rise to the patients' symptoms). In some cases, CNS drugs approved by the FDA (Food and Drug Administration) and targeted toward the relief of one CNS disorder, have been found to also relieve the symptoms of a different disorder. For example, a drug directed to relieve depression may help to relieve a different disorder (e.g., different diagnostic category) such as anxiety, posttraumatic stress disorder, etc. For second example, a drug directed at epilepsy may also relieve headaches and mania.

In a series of articles, Dr. Turan Itil has suggested the use of EEG (electroencephograph) measurements of the electrical signals from the brain as an aid in drug development. For example, he suggested the establishment of EEG patterns resulting from treatment with a first effective drug, and then testing a different drug, and determine if the patterns were similar. He postulated that a similar EEG pattern for the drug being tested may indicate that this drug may be effective for the same disorder as the original drug. An article by B. Saletu uses a different approach in which a diagnostic category, (e.g., depression) and a drug to be tested are selected. After administration of the drug under study, EEG recordings are taken from a normal group (without depression) and a study group (suffering from depression) and the EEG patterns from the two groups are compared to attempt to identify differences between the groups. Both of these approaches reportedly have had limited success, possibly because they are based on phenomenologic categories (diagnostic categories based on symptoms) and EEG recordings from the scalp, which do not localize the source of the recorded signals. However, there is room for much improvement.

### SUMMARY OF THE INVENTION

The present invention is directed to a method for determining effects of a drug on a central nervous system (CNS) of a subject, comprising the steps of: detecting and locating three-dimensional (3-D) brain source regions of first brain waves of the subject using a functional neuroimaging system, the first brain waves being indicative of a CNS disorder and comparing the first brain waves at each of the regions to control data corresponding to brain wave activity of corresponding brain regions in the absence of the CNS disorder. Then administering the drug to the subject, measuring second brain waves of the subject from the brain source regions and comparing the second brain waves to one of the first brain waves and the control data to determine whether the drug has had a desired effect on the CNS disorder.

### BRIEF DESCRIPTION OF THE DRAWINGS

For the purpose of illustration of the present invention, there are shown in the drawings forms which are presently preferred, it being understood, however, that the invention is not limited to the precise arrangements and instrumentalities which are shown:

FIG. 1 is a flow diagram of an exemplary method according to the present invention for investigation of a CNS drug; and

FIG. 2 is a schematic view of an exemplary embodiment of a system according to the present invention.

### DETAILED DESCRIPTION

In accordance with the present invention, there is provided a method and system to aid in investigating the effectiveness of drugs directed to the central nervous system (CNS) disorders. According to the present invention, an area of the brain which gives rise to the disorder, or which is effected by the drug being tested, is determined by source localization, preferably using Magnetoencephology (MEG).

Using one patient having a brain disorder as a test subject, the brain area of that patient is examined, e.g., by MEG, to locate an area giving rise to the disorder. Subsequently, a CNS drug is administered to the subject and its effects on the same brain area are determined by, e.g., MEG. If there is no change, or an undesired change, it is determined that the drug is ineffective.

An alternative exemplary procedure, in accordance with the present invention, using a patient with a CNS disorder as the subject, is to administer a CNS drug to the patient. Using MEG source localization, the patient's brain activity is monitored to locate any brain areas which show a change in activity in response to the drug.

The procedure, may preferably include the following three steps:

1. Determine whether a subject's brain has some abnormality. This may be independent of trying to place the patient in a diagnostic category based on the patient's symptoms;

2. Obtain a 3-D localization of the source of abnormality in the brain of the patient, for example using MEG. This will identify the location of the pathophysiology;

3. Administer a CNS drug to target the abnormal region;

4. Detect changes in the region (using, e.g., MEG) after administration of the CNS drug; and

5. Determine effectiveness of the drug based on changes in that region (i.e., reversal of pathophysiology in that region).

It may happen that a number of CNS drugs, each one directed to a specific and different pathophysiologies, may be used to treat a single disorder, (e.g., 2 or 3 drugs simultaneously given may be found to be most effective, for example, to treat anxiety). However, this method does not require the identification or use of a particular diagnostic category for the abnormality.

Magnetoencephalography (MEG) (sometimes called MSI-Magnetic Source Imaging) detects and measures magnetic components of brain wave signals (magnetic activity from neuronal firing) and, as these components, pass freely through the brain and skull and are not significantly attenuated or distorted by such passage, it is possible to obtain a 3-D (3 dimensional) localization of the source of magnetic signals. In addition, in MEG, the detection devices (superconducting coils of magnetometers) are connected to Superconductivity Quantum Interference Devices (SQUIDS) which are brought close to the patient's head without being attached thereto. This avoids several of the difficulties associated with the attachment of EEG electrodes to a patient. Those skilled in the art will understand that, compared to EEG devices, MEG devices are costly and, for example, require a magnetically shielded room which further adds to the cost.

The present invention will be explained primarily in terms of using MEG as a preferred system for 3-D brain source localization. However, it will be understood by those skilled in the art, that other 3-D localization systems may alternatively be used, including Low Resolution Brain Electromagnetic Topography (LORETA), Variable Resolution Electromagnetic Tomography (VARETA), Functional Magnetic Resonance Imaging (fMRI), Magnetic Resonance Spectroscopy, Positron Emission Tomography (PET) and Single Photon Emission Tomography (SPET).

The entire disclosures of U.S. Pat. No. 4,800,888 to T. M. Itil et al.; International Patent Publication WO 02/00110 A1 published on Jan. 3, 2002; U.S. Pat. No. 5,755,227 to Tomita et al; U.S. Pat. No. 6,370,414 to Robinson; and U.S. Pat. No. 4,913,152 describing various devices and methods for detecting and analyzing brain activity are hereby incorporated by reference herein.

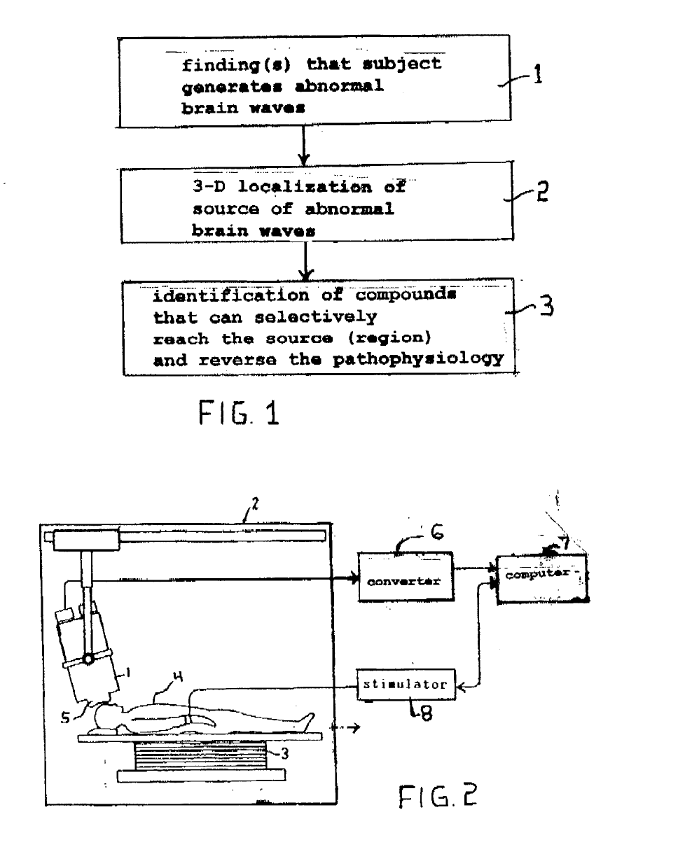

Preferred exemplary embodiments of present the invention will be described hereinafter with reference to the drawings. To investigate the effectiveness of a CNS drug, a subject is first evaluated to discover if his brain is generating abnormal brain waves. Step 1 of FIG. 1 may then be independent of a medical diagnosis of the subject. However, the effectiveness of the CNS drug (e.g., a drug to reduce the symptoms of depression) would be tested on a group of subjects having depression, the group being divided into control and test sub-groups.

In step 2, a source of abnormal brain waves is located by 3-D localization using functional neuroimaging (e.g., MEG). This identifies a region (set of 3-D voxels) which is the location of the subject's pathophysiology. As would be understood by those of skill in the art, a “voxel” is a three-dimensional space, (e.g., a 10 mm cube). It is quite possible that a single diagnostic category, (e.g., the category of depression), in a subject, may show a plurality of regions of abnormality.

In step 3, the drugs which may reach and positively affect the subjects brain regions of abnormality are identified. This may be determined by a comparison of the neuroimaging results of the brain region (or regions) being considered, before and after administration of the drug. If that comparison indicates an improvement (less abnormality of brain waves), it is an indication that the drug under investigation is effective.

There are a number of computer programs directed toward solving the problem of 3-D source localization as a relatively inexpensive alternative to MEG. One program is Low Resolution Brain Electromagnetic Topography (LORETA) as described by Pascal-Marqui et al., Japanese Journal of Clinical Neurophysiology 30: 81-94-2002. Another such computer program, software for source localization is Variable Resolution Electromagnetic Tomography (VARETA), see “3-D Statistical Parametric Mapping of EEG Source Spectra by means of Variable Resolution Electromagnetic Tomography, Clinical Electroencephalography; 32(2); 47-61, 2001. These articles are incorporated by reference herein.

As mentioned previously, a preferred method of brain source localization in neuroimaging is MEG which is discussed below. However, other methods of neuroimaging, such as VARETA, LORETA, fMRI (see U.S. Pat. No. 6,298,258), magnetic spectroscopy, Position Emission Tomography (PET) and single photon emission, may likewise be used. Both PET and FMRI provide 3-D images including information on metabolism, although temporal resolution is low compared to neuronal processes. These methods may generate functional images of the brain, both while the subject is resting and in an actuated state (in response to external stimuli).

There has been a considerable body of literature and patents directed toward the issue of how one distinguishes abnormal brain waves from normal brain waves. In general, the normality of brain waves is established by testing a group of “normal” subjects, who are free of symptoms of CNS disorders. Preferably, there are a series of normal groups, by age and gender, to establish a set of “normal” brain waves. In EEG, this procedure is referred to as Quantative Electroencephalography (QEEG) as described, e.g., in U.S. Pat. Nos. 5,699,808 and 6,016,444 which are expressly incorporated by reference herein. Many of the techniques of QEEG used to differentiate normal from abnormal brain waves may be applicable to this same problem using MEG although the QEEG techniques do not rely on source localization.

Some of the measures (e.g., parameters of functional mapping) which may be used to determine, using digital computer software programs, that a subject's brain waves are abnormal are the following:

1) relative power for each of the frequency domains (e.g., delta, theta, alpha, beta) from 0.5 to 50 Hz. including analysis by Fast Fourier Transforms (FFT), auto and cross-correlation functions, and pattern recognition of amplitude and frequency distributions;

2) coherence in each of the frequency domains;

3) symmetry in various frequency domains, the comparison being between the region suspected of being abnormal and other regions of the subject's brain believed to be normal;

4) the presence of abnormal activity (e.g., spikes, sharp waves, bursts, etc.) some of which may be characteristics of epilepsy; and

5) evoked responses (EP—evoked potential), and Averaged Evoked Potentials (AEP) to timed external stimulations including, for example, audio (e.g., clicks), visual (e.g., light flashes) and somasomatic (e.g., slight electrical shocks).

The information obtained from these measures, is then used to localize the sources of the abnormalities and may be combined with measures obtained from the measurement of metabolic activity using PET and/or SPECT. Once the source of the abnormal brain waves has been located one may, by administering an investigative program of CNS drug(s) try to regulate (normalize) that source. The objective is to find one or more drugs that move the identified abnormal region toward normality (normal space).

The drug (or drugs) being investigated should, of course, also be given to selected members of a normal group (people not showing symptoms of CNS disorders) to produce control information, using neurofunctional imaging on this normal group will provide control data indicative of whether the drug (or drugs) has an effect on regions of the brains of these normal subjects.

Many persons with CNS disorders manifest two or more abnormal symptoms (i.e., pathophysiologic markers). These patients may or may not show two or more brain regions of abnormality. Thus, in some cases it may be necessary to administer two or more drugs each of which is targeted to bring a specific region of abnormality into normality. Improvement in these regions, as determined by functional neuroimaging, may strongly correlate with the subjects clinical improvement (i.e., reduction of abnormal CNS symptoms).

FIG. 2 shows an exemplary embodiment of a system according to the present invention. As mentioned above, neuromagnetic brain signals from sources in the brain may be modeled as a current dipole which generates a magnetic field, in addition to the electrical field detected by an EEG.

The magnitude and direction of the magnetic field are detected and analyzed by the MEG. As shown in FIG. 2, the MEG 1 is contained within a magnetically shielded room 2. A bed 3 supports a subject 4. A multichannel SQUID sensor 5 which is positioned to scan the head of the subject 4 may be brought into approximation with the head of the patient, but need not come into contact therewith. The sensor 5 may include a plurality of magnetic sensors immersed in a cryogenic liquid coolant with a Dewyar (vacuum flask). Each sensor 5 has a pair of magnetic superconducting coils which detects magnetic field components radiated from the bipolar brain source; the coils are connected to Superconductivity quantum interference devices (SQUIDS) and the analog brain waves detected thereby are converted into digital data by an analog/digital converter 6. A control and analysis computer (CPU) 7 controls a stimulator 8 to generate stimulus for evoked responses (EP). The computer 7 collects and analyzes the data, as described above, under control of a suitable software program and prints it out or/and displays it on a monitor.

There are many modifications and variations of the above described illustrative embodiments which will be apparent to those skilled in the art without departing from the teaching of the invention. These modifications and variations are considered to be within the scope of the invention which is to be limited only by the claims appended hereto.

What is claimed is:

1. A method for determining effects of a drug on the central nervous system (CNS) of a subject, comprising the steps of:
(a) detecting and locating three-dimensional (3-D) brain source regions of first brain waves of the subject (patient) using a functional neuroimaging system, the first brain waves being indicative of a CNS disorder;
(b) comparing the first brain waves at each of the regions to control data corresponding to brain wave activity of corresponding brain regions in the absence of the CNS disorder;
(c) after steps (a) and (b), administering the drug to the subject (patient);
(d) measuring second brain waves of the subject (patient) from the brain source regions;
(e) comparing the second brain waves to one of the first brain waves and the control data; and
(f) determining whether the drug has had a desired effect on the CNS disorder based on the comparison of step (e).

2. The method as in claim 1, wherein the functional neuroimaging system is Magnetoencepholography (MEG).

3. A method as in claim 1, wherein the neuroimaging system is selected from a group of VARETA, LORETA, functional magnetic resonance imaging (fMRI), magnetic resonance spectroscopy, positron emission tomography (PET) and single photon emission tomography, (SPECT).

4. A method for determining effectiveness of a drug in treating a central nervous system (CNS) disorder, comprising the steps of:
analyzing brain activity of a subject to detect abnormal activity corresponding to the CNS disorder;
locating 3 dimensional source regions of the brain of the subject from which the abnormal activity originates;
administering the drug to the subject; and
analyzing brain activity of the subject in the source regions after administration of the drug to determine effectiveness of the drug.

5. A method as in claim 4, wherein the functional neuroimaging system is magnetoencepholography (MEG).

6. A method as in claim 4, wherein the neuroimaging system is selected from a group of VARETA, LORETA, functional magnetic resonance imaging (FMRI), magnetic resonance spectroscopy, positron emission tomography (PET), and single photon emission tomography (SPECT).

7. A system for determining effects of a drug on a central nervous system (CNS) of a subject evidencing a CNS disorder comprising:
(a) a functional neuroimaging system detecting and locating three dimensional (3-D) brain source regions of brain waves of the subject to generate subject brain wave data; and
(b) a computer analyzing comparing the subject brain wave data to control data corresponding to brain activity from comparative regions of a brain of a control subject not suffering from the CNS disorder to identify abnormal regions of the subject's brain generating abnormal brain waves, the computer then comparing brain activity after administration of the drug to one of the activity of the abnormal regions before administration of the drug and the control data.

8. A system as in claim 7, wherein the functional neuroimaging system is magnetoencepholography (MEG).

9. A system as in claim 7, wherein the neuroimaging system is selected from a group of VARETA, LORETA, functional magnetic resonance imaging (fMRI), magnetic resonance spectroscopy, positron emission tomography (PET), and single photon emission tomography (SPECT).
